(19) United States
(12) Patent Application Publication
(10) Pub. No.: US 2025/0367111 A1
(43) Pub. Date: Dec. 4, 2025

(54) DELIVERY OF SUSTAINED LOCAL AND SYSTEMIC IMMUNOMODULATION

(71) Applicants: Northeastern University, Boston, MA (US); The Brigham and Women's Hospital, Inc., Boston, MA (US)

(72) Inventors: Needa Brown, Orlando, FL (US); Srinivas Sridhar, Newton, MA (US); Jennifer Lynn Guerriero, Somerville, MA (US); Alexander P. Gottieb, Needham, MA (US)

(21) Appl. No.: 19/067,282

(22) Filed: Feb. 28, 2025

Related U.S. Application Data
(60) Provisional application No. 63/559,285, filed on Feb. 29, 2024.

(51) Int. Cl. A61K 9/00; A61K 31/353; A61K 31/4184; A61K 31/502; A61K 31/7016; A61K 31/7084; A61K 47/34; A61P 35/04

(52) U.S. Cl. CPC A61K 9/0024; A61K 31/353; A61K 31/4184; A61K 31/502; A61K 31/7016; A61K 31/7084; A61K 47/34; A61P 35/04

(57) ABSTRACT

Disclosed are sustained biodegradable implant (a depot) that releases high drug doses directly into the tumor for treating cancer. The depot comprises a biodegradable polymer and a STING agonist and/or a PARP inhibitor (PARPi); wherein the STING agonist or the PARPi is distributed in the biodegradable polymer. The depot activates anticancer innate and adaptive immunity within the tumor microenvironment and promote immune infiltration of secondary, metastatic sites.

### RELATED APPLICATIONS

This application claims the benefit of priority to U.S. Patent Application Ser. No. 63/559,285, filed Feb. 29, 2024.

### GOVERNMENT SUPPORT

This invention was made with government support under Grant Number HT9425-23-1-0274 and HT9425-23-1-0813 awarded by the Department of Defense/CDMRP. The Government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Jul. 25, 2025, is named NEX-17701_SL.xml and is 26,822 bytes in size.

### BACKGROUND

Immunotherapy is given either systemically via intravenous (i.v.) injections of antibodies or small molecules, which can lead to systemic inflammatory responses due to non-specific accumulation. Alternatively, direct tumor or disease site injections of drug have been pushed through to clinical investigation, but patients need to get direct tumor injections weekly for a periodic activation of the immune system. Accordingly, there is need for sustained formulations that allow for sustained delivery of immunomodulators.

### SUMMARY OF THE INVENTION

Disclosed is the use of nano-bio formulations that allow for slow release of low concentration immunomodulatory agents that trigger a continuous immune activation ranging from hours to days. This approach directs immunomodulation to the tumor or disease area of interest and decreases the number of times a patient would need to come to the clinic due to the longer release and modulation periods. Most continuous release is focused on molecular inhibitor molecule drugs but the idea of continuously modulating the immune system is novel.

In one aspect the present disclosure provides a depot comprising a biodegradable polymer and a STING agonist or a PARP inhibitor (PARPi). In some embodiments, the STING agonist or the PARPi is distributed in the biodegradable polymer. In some embodiments, the depot is configured to be implanted at a tumor site of a patient.

In some embodiments, the depot releases the STING agonist or the PARPi at the tumor site for a period of time. In some embodiments, the period of time is no less than 15 days. In some embodiments, the period of time is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. In some embodiments, the depot is configured to be delivered to the tumor site through a needle. In some embodiments, the needle is an 18G needle.

In some embodiments, the biodegradable polymer comprises polyglycolide (PGA), polycaprolactone (PCL), poly(DL-lactic acid) (PLA), poly(alpha-hydroxy acids), poly(lactide-co-glycolide) (PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or poly(DL-lactide-co-glycolide-co-caprolactone). In some embodiments, the polymer is poly(DL-lactide-co-glycolide) (PLGA). In some embodiments, the PLGA comprises equal parts lactide and glycolide. In some embodiments, said PLGA comprises 75% lactide and 25% glycolide, or 85% lactide and 15% glycolide.

In some embodiments, the depot comprises 1 mg to 10 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, 190 mg to 200 mg, 200 mg to 210 mg, 210 mg to 220 mg, 220 mg to 230 mg, 230 mg to 240 mg, 240 mg to 250 mg, 250 mg to 260 mg, 260 mg to 270 mg, 270 mg to 280 mg, 280 mg to 290 mg, 290 mg to 300 mg, 300 mg to 310 mg, 310 mg to 320 mg, 320 mg to 330 mg, 330 mg to 340 mg, 340 mg to 350 mg, 350 mg to 360 mg, 360 mg to 370 mg, 370 mg to 380 mg, 380 mg to 390 mg, or 390 mg to 400 mg of the STING agonist or the PARPi.

In some embodiments, the depot comprises both the STING agonist and the PARPi. In some embodiments, the STING agonist stimulates an anticancer immune microenvironment. In some embodiments, the STING agonist activates anticancer innate and adaptive immunity within the tumor microenvironment. In some embodiments, the STING agonist and the PARPi promote immune infiltration of secondary, metastatic sites. In some embodiments, the STING agonist amplifies the antitumor efficacy of the PARPi.

In some embodiments, the STING agonist is MK-1454, ADU-S100, GSK3745417, SB 11285, DMXAA, MPLA (Monophosphoryl Lipid A), a Cyclic Dinucleotide (CDN), E7766, BMS-986301, or Astin C. In some embodiments, the PARPi is Olaparib, Niraparib, and Talazoparib (TLZ), Rucaparib, Veliparib, Pamiparib, or Fuzuloparib. In some embodiments, the depot has a loading efficacy of 10, 20, 30, 40, 50, 60, 70, 80, 90, 100, 110, 120, 130, 140, 150, 160, 170, 180, 190, or 200 ug mm-1.

In another aspect the present disclosure provides methods of treating cancer in a subject in need thereof, comprising administering to a tumor site of the subject an effective amount of the depot described herein. In some embodiments, the depot is retained at the tumor site and exhibits sustained release of the STING agonist or the PARPi.

In some embodiments, the method further comprises administering an anti-PDL1 antibody. In some embodiments, the anti-PDL1 is formulated in a liposome. In some embodiments, the liposome comprises DSPE-PEG-amine (1,2-Distearoyl-sn-glycero-3-phosphoethanolamine-polyethylene glycol-amine) and/or DSPE-PEG-COOH (1,2-Distearoyl-sn-glycero-3-phosphoethanolamine-polyethylene glycol-carboxyl), DOTAP (1,2-Diolcoyl-3-trimethylammonium-propane), cholesterol, and DPPC (1,2-Dipalmitoyl-sn-glycero-3-phosphocholine). In some embodiments, the anti-PDL1 is administered every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, or every 20 weeks. In some embodiments, the liposome is 50 nm, 55 nm, 60 nm, 65 nm, 70 nm, 75 nm, 80 nm, 85 nm, 90 nm, 95 nm, or 100 nm in size.

In some embodiments, the anti-PDL1 antibody is atezolizumab, avelumab, durvalumab, or cosibelimab. In some embodiments, the anti-PDL1 antibody comprises a variable heavy chain complementarity determining region 1 (CDRH1), a variable heavy chain complementarity determining region 2 (CDRH2), a variable heavy chain complementarity determining region 3 (CDRH3), a variable light chain complementarity determining region 1 (CDRL1), a variable light chain complementarity determining region 2 (CDRL2), and a variable light chain complementarity determining region 3 (CDRL3); wherein
(a) (i) CDRH1 comprises the amino acid sequence of SEQ ID NO: 1;
   (ii) CDRH2 comprises the amino acid sequence of SEQ ID NO: 2;
   (iii) CDRH3 comprises the amino acid sequence of SEQ ID NO: 3;
   (iv) CDRL1 comprises the amino acid sequence of SEQ ID NO: 4,
   (v) CDRL2 comprises the amino acid sequence of SEQ ID NO: 5; and
   (vi) CDRL3 comprises the amino acid sequence of SEQ ID NO: 6, or
(b) (i) CDRH1 comprises the amino acid sequence of SEQ ID NO: 11;
   (ii) CDRH2 comprises the amino acid sequence of SEQ ID NO: 12;
   (iii) CDRH3 comprises the amino acid sequence of SEQ ID NO: 13;
   (iv) CDRL1 comprises the amino acid sequence of SEQ ID NO: 14,
   (v) CDRL2 comprises the amino acid sequence of SEQ ID NO: 15; and
   (vi) CDRL3 comprises the amino acid sequence of SEQ ID NO: 16, or
(c) (i) CDRH1 comprises the amino acid sequence of SEQ ID NO: 19;
   (ii) CDRH2 comprises the amino acid sequence of SEQ ID NO: 20;
   (iii) CDRH3 comprises the amino acid sequence of SEQ ID NO: 21;
   (iv) CDRL1 comprises the amino acid sequence of SEQ ID NO: 22,
   (v) CDRL2 comprises the amino acid sequence of SEQ ID NO: 23; and
   (vi) CDRL3 comprises the amino acid sequence of SEQ ID NO: 24.

In some embodiments, the anti-PDL1 comprises a combination of:
(a) a variable heavy chain comprising the amino acid sequence of SEQ ID NO: 7, and a variable light chain comprising the amino acid sequence of SEQ ID NO: 8, or
(b) a variable heavy chain comprising the amino acid sequence of SEQ ID NO: 17, and a variable light chain comprising the amino acid sequence of SEQ ID NO: 18, or
(c) a variable heavy chain comprising the amino acid sequence of SEQ ID NO: 25, and a variable light chain comprising the amino acid sequence of SEQ ID NO: 26, or
(d) a variable heavy chain comprising the amino acid sequence of SEQ ID NO: 27, and a variable light chain comprising the amino acid sequence of SEQ ID NO: 28.

In some embodiments, the antibody is a monoclonal antibody, a single chain antibody (scAb), a Fab fragment, a F(ab′)2 fragment, a single chain variable fragment (scFv), a scFv-Fc fragment, a multimeric antibody, or a bispecific antibody. In some embodiments, the antibody is a chimeric, humanized or fully human monoclonal antibody.

In some embodiments, the method further comprises administering a STING agonist or a PARPi. In some embodiments, the STING agonist or the PARPi is formulated in a liposome. In some embodiments, the liposome comprises DSPE-PEG-amine (1,2-Distearoyl-sn-glycero-3-phosphoethanolamine-polyethylene glycol-amine) and/or DSPE-PEG-COOH (1,2-Distearoyl-sn-glycero-3-phosphoethanolamine-polyethylene glycol-carboxyl), DOTAP (1,2-Diolcoyl-3-trimethylammonium-propane), cholesterol, and DPPC (1,2-Dipalmitoyl-sn-glycero-3-phosphocholine). In some embodiments the STING agonist or the PARPi is administered every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, or every 20 weeks. In some embodiments, the liposome is 50 nm, 55 nm, 60 nm, 65 nm, 70 nm, 75 nm, 80 nm, 85 nm, 90 nm, 95 nm, or 100 nm in size.

In some embodiments, the cancer is a solid tumor. In some embodiments, the cancer is breast cancer, ovarian cancer, or colon cancer. In some embodiments, the breast cancer is triple-Negative Breast Cancer (TNBC), advanced breast cancer. In some embodiments, the cancer is lung cancer, prostate cancer, colorectal cancer, pancreatic cancer, liver cancer, kidney cancer, cervical cancer, esophageal cancer, osteosarcoma, chondrosarcoma, liposarcoma, leiomyosarcoma, rhabdomyosarcoma, glioblastoma, meningioma, medulloblastoma, astrocytoma, ependymoma, Hodgkin lymphoma, or non-Hodgkin lymphoma.

In some embodiments, the depot is administered every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, or every 20 weeks.

### BRIEF DESCRIPTION OF THE DRAWINGS

FIG. 1 shows that optimal levels of sustained activation of the STING pathway using biodegradable implants stimulates anticancer innate and adaptive immunity and enhances the efficacy of PARPi for translation to advanced breast cancer patients.

FIGS. 2A-2E show sustained release implants. FIG. 2A show expected release profiles in vivo of various delivery methods: sustained release, a single dose, and multiple injections. Sustained release maintains drug concentration within the therapeutic window for an extended period. FIG. 2B show schematic of in vivo degradation of PLGA SMAART implants. SEM of implant highlighting a rough surface which is an indicator of drug loading. FIGS. 2C-2E show release profiles of three drugs (DTX-docetaxel, TLZ-talazoparib, DOC-doxorubicin) demonstrating the ease of fabrication of implants with various drugs, different tailored release profiles and a range of loading efficiencies (25-250 μg mm⁻¹). Differences in pH (physiological or tumoral) impacts release kinetics. For the same compound, drug loading does not impact release.

FIG. 3 shows that PARPi inhibits DNA damage repair (gray T-bar) which leads to release of chemokines and activation of immune cells. DNA damage 1) activates the cGAS-STING pathway leading to the release of interferon cytokines that stimulate antigen presenting cells such as dendritic cells and macrophage polarization, which can be amplified with exogeneous STING agonist and 2) induces expression of PDL1 to inhibit CD8+ T-cells activity, while addition of anti-PDL1 blocks PD-1/PDL1 interaction.

FIGS. 4A-4F show solvent evaporation method with 50:50 PLGA+ADU-S100 sodium. FIG. 4A shows drug loading of first generation SMAART-STING implants at three different weight ratios confirms linearity of final loading compared to initial polymer: drug ratio using 50:50 PLGA and ADU-S100 disodium salt. FIG. 4AB shows encapsulation efficacy of SMAART implants decreased with increasing ratios of polymer: drug due to increase in suspension viscosity. FIG. 4C shows SEM of a blank 50:50 PLGA implant. FIG. 4D shows THP-1 reporter cell line confirms that implant loaded ADU-S100 maintains bioactivity and this activity is STING specific (H-151, STING inhibitor). FIG. 4E shows SMAART-STING sustained release kinetics in PBS (pH 7.4, 37° C.). FIG. 4F shows released drug at day 11 maintains bioactivity.

FIGS. 5A-5D show solvent evaporation method with 75:25 PLGA+ADU-S100 sodium/ammonium. Solvent evaporation method-Loading map (1 mg start). If 100% loading=8.33 μg/mm. Actual loading 35.53%=>2.961+/−0.212 μg. Solvent evaporation method-Loading map (8 mg start-scale-up). If 100% loading=66.67 μg/mm. Actual loading 29.93%=>19.183+/−0.535 μg. FIG. 5A shows implanting mapping example of extruded tubing cut and selected pieces were evaluated via HPLC for homogeneity. FIG. 5B shows solvent evaporation implant formulation mapping for SMAART-STING using 75:25 ratio PLGA and ADU-S100 enantiomer ammonium salt. FIG. 5C shows sustained release of 75:25 PLGA SMAART-STING loaded with ADU-S100 enantiomer ammonium salt in PBS (pH 7.4, pH 6 and 37° C.). FIG. 5D shows drug loading of third generation SMAART-STING implants at two different weight ratios confirms linearity of final loading compared to initial polymer: drug ratio using 75:25 PLGA and ADU-S100 enantiomer ammonium salt.

FIGS. 6A-6C show hot melt extrusion method with 75:25 PLGA+ADU-S100 sodium/ammonium. Solvent evaporation method-Loading map (1 mg start). If 100% loading=8.33 μg/mm. Actual loading 35.53%=>2.961+/−0.212 μg. Solvent evaporation method-Loading map (8 mg start-scale-up). If 100% loading=66.67 μg/mm. Actual loading 29.93%=>19.183+/−0.535 μg. FIG. 6A shows hot melt extrusion implant formulation mapping for SMAART-STING using 50:50 ratio PLGA and ADU-S100 enantiomer ammonium salt. FIG. 6B shows sustained release of 50:50 PLGA SMAART-STING loaded with ADU-S100 enantiomer ammonium salt in PBS (37° C.) at pH 6 and pH 7.4. SE=solvent evaporation sample. Sample X=individual hot-melt extrusion samples. FIG. 6C shows drug loading of third generation SMAART-STING implants at two different weight ratios confirms linearity of final loading compared to initial polymer: drug ratio using 75:25 PLGA and ADU-S100 enantiomer ammonium salt via hot melt extrusion.

FIG. 7 shows solvent evaporation method with 75:25 PLGA+ADU-S100 sodium. In vitro sustained release of 75:25 PLGA:ADU-S100 implants via solvent based evaporation at pH 6 and pH 7 media in vitro. Additional analysis of media showed a change of pH during the hydrolysis and degradation of implant over time. In vivo release study in healthy mice with a subcutaneous implant placed over 21 days showed a zero-order release kinetics.

FIG. 8 shows THP-1 reporter cell line confirms that implant loaded ADU-S100 maintains bioactivity and this activity is STING specific (H-151, STING inhibitor).

FIGS. 9A-9B show that a TLZ dose study was conducted to identify a drug dose that would allow for the assessment of combinatorial approaches with soluble or SMARRT formulated ADU-S100 with TLZ shown as either FIG. 9A tumor volume or FIG. 9B overall survival.

### DETAILED DESCRIPTION

Triple-negative breast cancer (TNBC) comprises 15-20% of all breast cancers with limited treatment options. Poly-(ADP-ribose) polymerase inhibitors (PARPi) have emerged as a promising therapy for these patients; however, PARPi monotherapy has not proven to be a durable option, necessitating research for rapid and durable translational strategies. Enlisting the patient's adaptive immunity via immune checkpoint blockade (ICB) has rapidly gained momentum with the first ICB FDA approved in 2011. Although promising, only a portion of patients benefit from the addition of ICB. Enhancing T-cell infiltration and activation has been the focus of ICB, however the largest population of tumor infiltrating immune cells are macrophages, which are the link between the innate and adaptive immune systems. PARPi remodels innate immunity, promoting deleterious tumorigenic tumor associated macrophages (TAMs) that inhibit T-cell activation and function, preventing therapeutic responses. Targeting of the cGAS/STING pathway with exogenous STING agonist is an innate immune modulatory therapy to reprogram TAMs. Delivery of intratumoral STING agonist in combination with the PARPi, olaparib, achieved complete regression in BRCA-deficient TNBC models.

A limitation to translation of STING agonist is the necessity of repeated intratumoral injections to achieve high disease site drug concentration while avoiding systemic inflammatory responses. Provided herein is a sustained biodegradable implant that can linearly release high drug doses directly into the tumor over a 28-day period, providing the missing platform necessary for clinical translation of STING agonist.

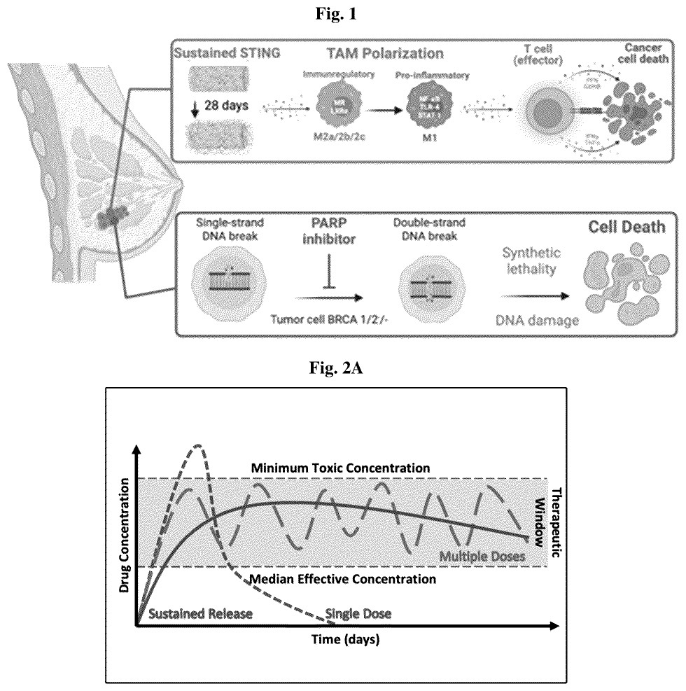

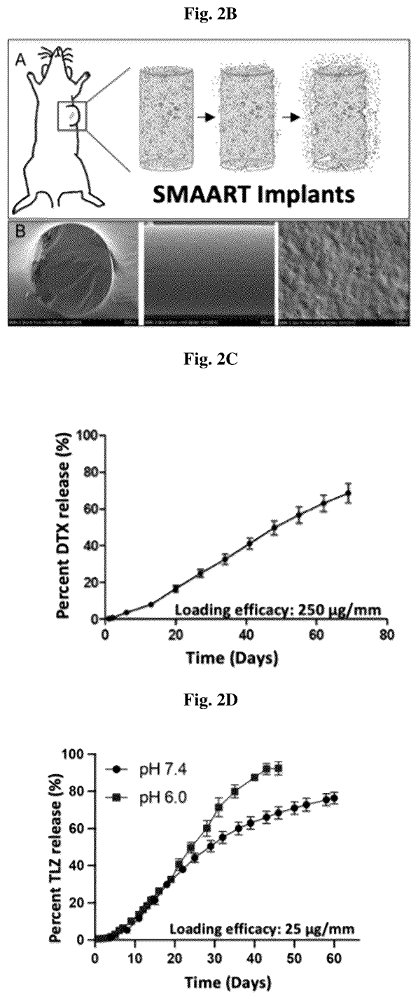

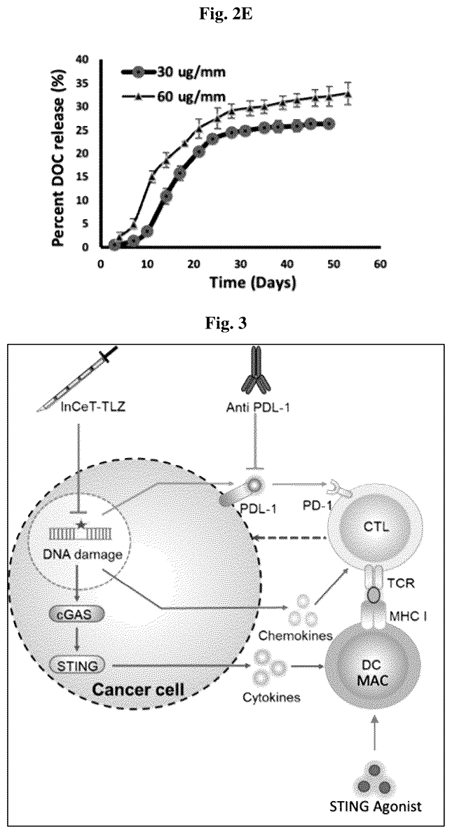

The efficacy of sustained STING agonist implants are tested to activate innate and adaptive immunity compared to periodic injections. Sustained STING pathway activation optimally stimulates an anticancer immune microenvironment within the primary site and in combination with PARPi promote immune infiltration of secondary, metastatic sites (FIG. 1). The preclinical data generated provide evidence for translation of STING agonist implants to overcome barriers for continuous delivery.

Sustained STING agonist delivery activates innate and adaptive anticancer immunity at primary and secondary, metastatic sites that eradicate advanced breast cancer in combination with PARPi therapy as demonstrated in sophisticated, patient-mimicking genetically engineering mouse models (GEMM) of breast cancer. This formulation revolutionizes treatment regimens by replacing them with ones that are more effective, less toxic, and impact survival and eliminate the mortality associated with metastatic breast cancer.

The highly efficacious combination therapy disclosed herein treats cancer (e.g., advanced breast cancer) while minimizing systemic toxicity and reducing patient burden. Sustained activation of the STING pathway using biodegradable STING agonist implants activates anticancer innate and adaptive immunity within the tumor microenvironment compared to periodic STING agonist injections. Sustained STING pathway modulation amplifies the antitumor efficacy of PARPi therapy at primary and secondary, metastatic sites for rapid translation to advanced breast cancer patients.

Sustained release STING agonist implants are formulated to improve tumoral delivery and immunity compared to periodic agonist injections while limiting systemic toxicity. The therapeutic impact of sustained STING activation and PARPi at primary and secondary, metastatic sites for rapid translation to breast cancer patients is assessed. Provided herein is the platform necessary to translate STING agonist into the clinic by overcoming barriers for continuous delivery. A novel, biodegradable platform is formulated for sustained intratumoral STING activation that is the crucial step necessary for clinical success of PARPi strategies to eradicate advanced breast cancer.

Poly-(ADP-ribose) polymerase (PARP) are crucial for recognizing single-stranded breaks (SSBs) and triggering a DNA repair cascade. PARP binds to SSB sites, recruits DNA repair machinery, and catalyzes autoPARPylation that in turn leads to the release of PARP from the damaged site. PARP inhibitors (PARPi) prevent the release of PARP by inhibiting autoPARPylation and trapping the PARP-DNA complex which can lead to stalled replication forks, persistent SSBs, and eventually double stranded breaks (DSBs). Cells rely on homologous repair (HR) DSB pathways which are mediated by BRCA1 and BRCA2. BRCA ½-deficient cancers, such as ovarian and breast, are highly sensitive to PARPi therapy leading to synthetic lethality.

Combinatorial approaches focusing on cGAS-STING activation, M1 polarization, and PDL1 receptor blocking have been effectively synergized with PARPi and are being studied clinically. Although PARPis, Olaparib, Niraparib, and Talazoparib (TLZ), as single agents increased expression of PDL1 in breast and ovarian cancers, the combination of Olaparib or Niraparib and anti-PDL1 antibodies slowed tumor growth and restored CD8+ T-cell infiltration, indicating a promising therapeutic combination with anti-PDL1. Focus on CD8+ T-cell activation has gained interest as a complementary mechanism for tumor regression; however, the largest population of infiltrating immune cells are macrophages that link the innate and adaptive immune system. Recent work in BRCA^{-/-} breast cancer exploring synergistic approaches to PARPi resistant tumors has focused on reprogramming macrophages from a pro-tumor M2 to an anti-tumor M1 phenotype using a STING agonist. STING activation has been shown to be crucial for PARPi, olaparib, anti-tumor immune response in a BRCA1^{-/-} ovarian cancer. This shift in macrophage polarization has been linked to improve PARPi response and overall survival in a CD8+ T-cell dependent manner.

Ovarian cancer clinical trials evaluating combinations with PARPi have been limited due to the highly toxic myelosuppression following systemic PARPi. More than 50% of ovarian tumors have a defective HR DNA repair pathway, making these tumors prime candidates for PARPi. In the ENGOT-OV16/NOVA clinical trial, PARPi doses needed to be reduced in 68.9% of patients due to treatment-emergent adverse events (TEAEs) with a discontinuation rate of 14.7%. In a recent phase II clinical trial with systemic PARPi Olaparib and anti-PDL1, all patients had at least one grade of hematologic toxicity and 31% of patients had grade ≥3 anemia. In the MEDIOLA trial, combination of Olaparib and anti-PDL1 led to 65.5% disease control but 17.6% experienced grade ≥3 anemia.

TLZ is a PARPi which has about 100-fold higher lethality compared to Olaparib, Rucaparib, and Niraparib. Preclinical work has shown promise in TLZ-induced DNA damage and STING immunomodulatory activation regardless of BRCA mutations in ovarian and colon cancers thus expanding the patient population for TLZ compared to other PARPi. But the extension of TLZ to other malignancies has been hindered due to the more potent side effects resembling chemotherapeutics. Due to its higher potency and ability to trap PARP, TLZ is recommended at a significantly lower dose of 1 mg daily compared to 300 mg or more for other PARPi. Current oral delivery mode of TLZ has a significant rate limiting factor, the gastrointestinal absorption barrier which necessitates higher doses of TLZ to achieve tumor specific therapeutic levels. In the EMBRACA clinical trial, adverse events were identified in 65% of TLZ patients, leading to a dose reduction below the therapeutic level for 53% of those patients. Given the high potency of TLZ, the sustained delivery of a TLZ implant (InCeT-TLZ) is studied to release TLZ over several days directly to the peritoneal cavity to treat metastatic ovarian cancer (mOC) and not distribute systemically, thus minimizing severe side effects.

Described herein are effective combinations of sustained release InCeT-TLZ with immunomodulatory factors, anti-PDL1 and STING agonist, to synergize with PARPi and minimize systemic toxicity in a patient-mimicking, peritoneal metastatic high grade serous ovarian cancer model. Here we propose a unique, dual innate and adaptive modulatory nanoformulation combining a liposomal formulation of STING agonist, ADU-S100, which is currently in clinical trials, with anti-PDL1 targeting as a multi-pronged approach for PARPi synergy. Our approach with sustained PARPi delivery within the peritoneal cavity limits systemic toxicity, allowing for more patients to benefit from immune modulatory combinations.

TLZ based polymeric implants (InCeT-TLZ), for localized delivery, were synthesized using Poly(lactic-co-glycolic) acid (PLGA) as a matrix polymer, following the solvent evaporation-based protocol previously developed (Kumar, R., et al., Nanoparticle-Based Brachytherapy Spacers for Delivery of Localized Combined Chemoradiation Therapy. International Journal of Radiation Oncology Biology Physics, 2015. 91 (2): p. 393-400; Tangatoori, S. S. S., Nanoparticle drug delivery system and method of treating cancer and neurotrauma. US PCT/US2014/0536 March 2015; Belz, J. E., et al., Sustained Release Talazoparib Implants for Localized Treatment of BRCA1-deficient Breast Cancer. Theranostics, 2017. 7 (17): p. 4340-4349). The implant was designed to allow direct placement into peritoneal cavity using an 18G needle (no surgery required), after which TLZ was slowly released from InCeT-TLZ due to PLGA degradation primarily by hydrolysis and allowed to diffuse into the tumor. Here we evaluated the release kinetics of 1 and 2 mm InCeT-TLZ implants, loaded with 25 μg and 50 μg of TLZ respectively, and showed similar sustained release kinetics with close to 100% of the drug released over 30 days.

No toxicity was identified with InCeT-TLZ compared to oral TLZ. PARPi have been limited due to induction of bone marrow toxicity. Histopathology was analyzed in mFT3666 mOC mice following peritoneal InCeT-TLZ implantation and no toxicity was identified. Using a Procyte Dx Analyzer, decrease in red blood cells in BRCA^{-/-} breast cancer mice was observed following oral TLZ indicative of clinical bone marrow toxicity.

InCeT-TLZ favorably alters cancer biomarkers compared with systemic administration or empty implants. Mammary glands were harvested 4 weeks after BRCA1-deficient mice were treated with implants (InCeT-TLZ or empty InCeT) or free TLZ. As shown in, over half of the epithelial cells in the mammary glands of mice treated with empty InCeT were positive for PCNA, confirming robust proliferation. Treatment with InCeT-TLZ and free TLZ significantly reduced PCNA+ cells by ˜50%. InCeT-TLZ but not free TLZ increased γ-H2AX expression, a marker of dsDNA breaks and PARPi efficacy, which leads to synthetic lethality in BRCA1-deficient tumors.

Sustained PARP inhibition doubles survival in BRCA^{-/-} mOC model. Therapeutic efficacy was tested in vivo in a BRCA^{-/-} model with conditional BRCA1/2, PTEN, and TP53 deletions (mFT3666) that mimics late stage disseminated disease. Free TLZ slowed disease progression and extended median survival to ˜70 days, compared with control (˜57 days). These animals eventually developed severe ascites and had to be sacrificed. A InCeT-TLZ implant loaded with 50 μg of TLZ was injected into the peritoneal cavity using an 18 gauge needle every 25 days. Most of the InCeT-TLZ group of animals survived until 93 days, with some developing ascites.

InCeT-TLZ administered as a sustained release implant intraperitoneally minimizes toxicities to non-target organs, improves drug bioavailability, delays or suppresses tumor progression, and allow for safer combinatorial treatment options. The mechanism of action of this dual therapy is depicted in and contains the following elements: (i) PARPi blocks SSB repair and (ii) in BRCA-deficient tumors leads to formation of DSBs and synthetic lethality. (iii) PARPi enhances infiltration of immune cells into the tumor microenvironment leading to increased anti-tumor activity through generation of proinflammatory cytokines through the cGAS-STING pathway which (iv) can be further amplified via addition of exogenous STING agonist; (v) PARPi can also enhance PDL1 expression and hence immunosuppression, however immune checkpoint inhibitors (anti-PDL1) restores the CD8+ T cells and re-sensitize tumor cells to PARPi.

In one aspect the present disclosure provides a depot comprising a biodegradable polymer mixed with a STING agonist or a PARP inhibitor (PARPi). In another aspect the present disclosure provides methods of treating cancer in a subject in need thereof, the method comprises administering to a tumor site of the subject an effective amount of the depot described herein.

### Definitions

For convenience, certain terms employed in the specification, examples, and appended claims are collected here.

As used herein, the singular forms “a,” “an” and “the” are intended to include the plural forms as well, unless the context clearly indicates otherwise.

The term “about” means within an acceptable error range for the particular value as determined by one of ordinary skill in the art, which will depend in part on how the values measured or determined, i.e., the limitations of the measurement system. Where the terms “about” or “approximately” are used in the context of compositions containing amounts of ingredients or conditions such as temperature, these values include the stated value with a variation of 0-10% around the value (X±10%).

The terms “including,” “includes,” “having,” “has,” “with,” or variants thereof are inclusive in a manner similar to the term “comprising.” The term “consisting” and the grammatical variations of consist encompass embodiments with only the listed elements and excluding any other elements. The phrases “consisting essentially of” or “consists essentially of” encompass embodiments containing the specified materials or steps and those including materials and steps that do not materially affect the basic and novel characteristic(s) of the embodiments.

Ranges are stated in shorthand to avoid having to set out at length and describe each and every value within the range. Therefore, when ranges are stated for a value, any appropriate value within the range can be selected, and these values include the upper value and the lower value of the range. For example, a range of two to thirty represents the terminal values of two and thirty, as well as the intermediate values between two to thirty, and all intermediate ranges encompassed within two to thirty, such as two to five, two to eight, two to ten, etc.

The term “preventing” is art-recognized, and when used in relation to a condition is well understood in the art, and includes administration of a composition which reduces the frequency of, or delays the onset of, symptoms of a medical condition in a subject relative to a subject which does not receive the composition. Thus, prevention of cancer includes, for example, reducing the incidence of cancer in a population of patients receiving a prophylactic treatment relative to an untreated control population, and/or delaying the onset of cancer in a treated population versus an untreated control population, e.g., by a statistically and/or clinically significant amount.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment.

As used herein, a “depot” comprises a composition configured to administer at least one therapeutic agent to a tumor site in the body of a patient in a controlled, sustained manner. The depot also comprises the therapeutic agent itself. A depot may comprise a physical structure or carrier to configured to perform or enhance one or more functions related to treatment, such as facilitating implantation and/or retention in a treatment site (e.g., at or proximate a tumor), modulating the release profile of the therapeutic agent, increasing release towards a treatment site, reducing release away from a treatment site, or combinations thereof. In some embodiments, a “depot” includes but is not limited to rods, discs, films, sheets, strips, ribbons, capsules, coatings, matrices, wafers, pills, pellets, or other pharmaceutical delivery apparatus or a combination thereof. Moreover, as used herein, “depot” may refer to a single depot, or may refer to multiple depots. As an example, the statement “The depot may be configured to release 2 g of therapeutic agent to a treatment site” describes (a) a single depot that is configured to release 2 g of therapeutic agent to a treatment site, and (b) a plurality of depots that collectively are configured to release 2 g of therapeutic agent to a treatment site.

### Formulations

Formulation of immunomodulators for sustained continuous delivery:
1) A solvent-based evaporation method to fabricate implants, specifically, PLGA and a STING agonist is mixed in chloroform and then extruded through a 0.8 mm tubing. The tubing is dried overnight in an oven to evaporate the chloroform, leaving implants of PLGA and STING agonist.
2) A hot melt extrusion method to fabricate implants, specifically, PLGA and a STING agonist are mixed together, melted with heat and then extruded through a 0.8 mm tubing. The tubing is allowed to cool leaving implants of PLGA and STING agonist.
3) Lipid based nanoformulations of STING agonist using DSPE-PEG-amine and/or DSPE-PEG-COOH, DOTAP, cholesterol, and DPPC are formulated to encapsulate STING agonist for systemic delivery using microfluidics and/or thin film hydration method. Surface of the lipid-based nanoparticle can be modified with antibodies and/or biologics for disease specific targeting and/or adaptive immune modulation by attaching anti-PDL1, anti-CTLA4, anti-CSFR1, anti-OX40, anti-CD47, or other immunomodulatory antibodies.
*STING agonist can be replaced by other small molecule immunomodulatory agents.
*DOTAP and DPPC can be replaced by other equivalent lipids.

This formulation will, for first time, provide the following advantages:
1) Biodegradable components allow for sustained, continuous modulation (activation and/or suppression) of the immune system.
2) Local disease site delivery limits systemic exposure and systemic inflammatory responses.
3) Composition can be modulated to tailor release profile, time of sustained immune modulation, drug of immune modulation, and dose of immune modulation.
4) Easily modified to include other drugs, e.g. other small molecule immunomodulators.
5) Can be combined with other conventional i.v. chemotherapeutic administration (e.g. docetaxel, platinum-based, etc.) or oral (p.o.) administration (e.g. PARPi, etc.) so that existing therapeutic approaches are not compromised.
6) Lipid based nanoparticles allow for systemic administration to preferentially treat primary and metastatic disease sites at once.
7) Lipid formulations can be surface modified to increase targeting and combine innate and adaptive immune system modulation in one therapy (e.g. anti-PDL1, anti-CTLA4, anti-CSFR1, anti-OX40, anti-CD47, or other immunomodulatory antibodies).

This formulation will, for first time, further provide the following advantages:
1) Overcomes limits to systemic immune inflammatory responses.
2) Minimize patient burden-minimize necessary injections.
3) All polymers/lipids/drugs used at either FDA approved or under clinical investigation separately.
4) Formulations can be easily combined with conventional i.v. or p.o. chemotherapeutic administration.
5) Drug loading and release can be tailored to enable a broad dynamic range of drug delivery from several μg to mg.
6) Easily modified to include other drugs, e.g. other small molecule immunomodulators.
7) Lipid formulations can be surface modified to increase targeting and combine innate and adaptive immune system modulation in one therapy.
8) Local sustained, continuous immunotherapy to solid tumors and metastatic peritoneal tumors.
9) Sustained, continuous immunomodulator delivery to other autoimmune diseases, such as corticosteroids in biodegradable implants for local, low dose sustained arthritis treatment.
10) Nanoformulations allow for the preferential uptake by primary/metastatic tumor or diseased sites.
11) Can be implanted during surgery to treat micrometastatic disease with localized, sustained immunotherapy.
12) Decrease financial costs from repeat injections, continuous hospital visits, and failure of drug response due to poor patient compliance.
13) Delivery of immunomodulators for other autoimmune diseases (lupus, arthritis, etc.).

Disclosed herein are implantable depots and associated devices, systems, and methods for treating cancer via sustained, controlled release of a locally acting therapeutic agent (e.g., STING agonist and/or PARPi) while the depot is implanted at a tumor site in vivo.

As is understood in the art, “release” of the therapeutic agent includes movement of the therapeutic agent away from the depot, as well as the sustained presence of the therapeutic agent at the tumor site following implantation of the depot, regardless of the relative movement of the therapeutic agent with respect to the confines of the depot. Thus, any therapeutic agent that remains substantially stationary relative to its position when first implanted is still considered “released” so long as it provides a therapeutic benefit at the tumor site.

The depots of the present technology are configured to deliver a high, sustained local dose to cancer tissue over the course of days, weeks, or months. The depots may provide a high local concentration of therapeutic agent over a sustained period of time sufficient to cause toxicity of cancerous or neoplastic tissue while avoiding toxic exposure outside of the targeted tissue and, particularly, avoiding toxic exposure to the aforementioned critical, non-target structures. This pharmacokinetic profile may optimize treatment of the cancer while minimizing complications.

The depot may be configured to be implanted at a tumor site of a patient and releases the STING agonist or the PARPi at the tumor site for a period of time, such as no less than 15 days, preferably, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. In some embodiments, the depot forms an in-situ implant in a subject in need thereof following administration.

In some embodiments, the biodegradable polymer comprises polyglycolide (PGA), polycaprolactone (PCL), poly(DL-lactic acid) (PLA), poly(alpha-hydroxy acids), poly(lactide-co-glycolide) (PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or poly(DL-lactide-co-glycolide-co-caprolactone).

In various embodiments, the molecular weight of the polymer can be a wide range of values. The average molecular weight of the polymer can be from about 1000 to about 10,000,000 Da; or about 1,000 to about 1,000,000; or about 5,000 to about 500,000; or about 10,000 to about 100,000; or about 20,000 to 50,000, more preferably 7,000 to about 17,000.

In some embodiments, a ratio of the mass of the therapeutic agent in the depot to the polymer mass of the depot is at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, or at least 16:1. In some embodiments, the depot comprises 1 mg to 10 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, 190 mg to 200 mg, 200 mg to 210 mg, 210 mg to 220 mg, 220 mg to 230 mg, 230 mg to 240 mg, 240 mg to 250 mg, 250 mg to 260 mg, 260 mg to 270 mg, 270 mg to 280 mg, 280 mg to 290 mg, 290 mg to 300 mg, 300 mg to 310 mg, 310 mg to 320 mg, 320 mg to 330 mg, 330 mg to 340 mg, 340 mg to 350 mg, 350 mg to 360 mg, 360 mg to 370 mg, 370 mg to 380 mg, 380 mg to 390 mg, or 390 mg to 400 mg of the STING agonist or the PARPi.

The PLGA ratio influences crystallinity, solubility, rate of degradation and drug release. For example, the higher the lactide content, slower is the degradation vis-à-vis drug release. Poly lactic acid contains an asymmetric α-carbon which is typically described as the D or L form in classical stereochemical terms and sometimes as R and S form, respectively. The enantiomeric forms of the polymer PLA are poly D-lactic acid (PDLA) and poly L-lactic acid (PLLA). PLGA is generally an acronym for poly D,L-lactic-co-glycolic acid where D- and L-lactic acid forms are in equal ratio. The physicochemical properties of optically active PDLA and PLLA are nearly the same. In general, the polymer PLA can be made in highly crystalline form (PLLA) or completely amorphous (PDLA) due to disordered polymer chains. PGA is void of any methyl side groups and shows highly crystalline structure in contrast to PLA.

The term “biodegradable” as used herein refers to a component which erodes or degrades at its surfaces over time due, at least in part, to contact with substances found in the surrounding tissue fluids, or by cellular action.

Examples of STING agonists are MK-1454, ADU-S100, GSK3745417, SB 11285, DMXAA, MPLA (Monophosphoryl Lipid A), a Cyclic Dinucleotide (CDN), E7766, BMS-986301, and Astin C. Examples of the PARPi are Olaparib, Niraparib, and Talazoparib (TLZ), Rucaparib, Veliparib, Pamiparib, and Fuzuloparib.

In some embodiments, the depot further comprises a solvent, such as a biocompatible solvent. The biocompatible solvent can be a biocompatible polar aprotic solvent. In some embodiments, the solvent is miscible to dispersible in aqueous medium or body fluid. Suitable polar aprotic solvents are well known in the art and fully described in, for example, in Aldrich Handbook of Fine Chemicals and Laboratory Equipment, Milwaukee, Wis. (2000) and U.S. Pat. Nos. 6,565,875, 5,324,519; 4,938,763; 5,702,716; 5,744, 153; and 5,990,194, which are incorporated by reference herein in their entirety. In some embodiments, the solvent is capable of diffusing into body fluid so that the flowable composition coagulates or solidifies. In some embodiments, the solvent is biodegradable. In some embodiments, the solvent is non-toxic. As set forth in U.S. Pat. No. 6,565,875, examples of suitable polar aprotic solvents include polar aprotic solvents having an amide group, an ester group, a carbonate group, a ketone, an ether, a sulfonyl group, or a combination thereof.

In some embodiment, the polar aprotic solvent is N-methyl-2-pyrrolidone, 2-pyrrolidone, N, N-dimethylformamide, N, N-dimethylacetamide, dimethyl sulfoxide, propylene carbonate, caprolactam, triacetin, or any combination thereof. In some embodiments, the polar aprotic solvent is N-methyl-2-pyrrolidone. As set forth in U.S. Pat. No. 6,565,875, the polar aprotic solvent can be present in any suitable amount. The type and amount of biocompatible polar aprotic solvent present in the composition may depend upon the desired properties of the controlled release implant. In some embodiments, the type and amount of biocompatible polar aprotic solvent can influence the length of time in which the therapeutic agent is released from the controlled release implant. In some embodiments, an implant is formed in situ by the process of injecting the depo to a subject; allowing the solvent, in said depot, to dissipate to produce a solid biodegradable implant.

In some embodiments, the invention relates to a method of forming an implant in situ in a subject, the method comprising the steps of: injecting the depot to a subject; allowing the solvent, in said depot, to dissipate to produce a solid biodegradable implant.

### Method of Treatment

Triple-negative breast cancer (TNBC) is the most aggressive form of breast cancer with a higher risk of recurrence and worse prognosis after relapse than hormone receptor positive cancers (median survival 12-18 months vs. 50-60 months). Until recently conventional treatment options for TNBC patients involved either a mastectomy or a lumpectomy followed by radiation and/or chemotherapy. When given the option for breast conservation therapy (BCT), 72% of patients preferred some form of BCT over surgical options, especially younger women. In January 2018 the FDA approved the PARPi olaparib as the first targeted therapy for patients with TNBC, in which olaparib prolonged progression-free survival (PFS) compared to standard chemotherapy (7 vs. 4.2 months) and doubled overall response rate (ORR) to 59.9%. This was shortly followed by the approval of another PARPi, talazoparib (TLZ). PARP are crucial for recognizing single-stranded breaks (SSBs) and triggering a DNA repair cascade. PARP binds to SSB sites, recruits DNA repair machinery, and catalyzes autoPARPylation that in turn leads to the release of PARP from the damaged site. PARPi prevent the release of PARP by inhibiting autoPARPylation and trapping the PARP-DNA complex which can lead to stalled replication forks, persistent SSBs, and eventually double stranded breaks (DSBs). Cells rely on homologous recombination (HR) DSB repair pathways which are mediated by breast cancer genes, BRCA1 and BRCA2. BRCA 1/2-deficient cancers, such as breast and ovarian, are highly sensitive to PARPi therapy leading to synthetic lethality. PARPi are therefore a powerful anti-cancer therapy for TNBC; however, resistance emerges, and responses are not durable. Combinatorial approaches that augment the rate and durability of PARPi responses could transform the treatment of TNBC patients.

Immune checkpoint blockade (ICB) has revolutionized the treatment of some cancer types but has demonstrated only modest benefit in TNBC as a monotherapy. ICB has been recently FDA approved for TNBC in combination with chemotherapy, however the approval is only for PD-L1-positive metastatic TNBC and while there are responses, the majority of patients eventually progress. The limited response rates (5-20%) suggest that tumor-related immunosuppression cannot be fully overcome by ICB blockade alone. Thus far, two clinical studies have reported results of PARPi plus ICB in advanced breast cancer. In the TOPACIO study, 55 patients with advanced TNBC received the PARPi niraparib with pembrolizumab (anti-PD-1), irrespective of BRCA mutation status or PD-L1 expression, with an ORR of 21%, mainly driven by patients with BRCA mutations (ORR of 47% within BRCA-associated cohort). The MEDIOLA study was an open-label, phase ½, basket trial of the PARPi olaparib with durvalumab (anti-PD-L1) in solid tumors. Of patients enrolled in the BRCA-mutated breast cancer cohort, ORR was 63.3% and PFS was 8.2 months. Clinical outcomes of these two trials were similar to the results of the OlympiAD trial (ORR 59.9%, PFS 7.0 months).

T-cell infiltration and activation has been the focus of ICB; however, the largest population of infiltrating immune cells are tumor associated macrophages (TAMs) that link innate and adaptive immunity. Tumor immunophenotyping has revealed that TAMs can account for up to 50% of the breast tumor, and are generally suppressive and associated with increased vascular density, resistance to chemotherapy, and worse clinical outcomes.

As described herein, PARPi drives development of a subset of suppressive TAMs that restrict antitumor T-cell function. In the absence of TAMs, PARPi induce robust recruitment of cytotoxic T cells and durable antitumor responses. This work establishes TAMs as targets for anti-cancer therapy however TAM-targeting strategies have been slow to be approved for clinical care, likely due to lack of clinical rationale and testing of appropriate combinations.

Cyclic GMP-AMP Synthase-Stimulator of Interferon Genes (cGAS/STING) is a cytosolic DNA-sensing pathway that activates release of type I interferon (IFN) and other inflammatory cytokines regulating innate immune cells such as macrophages. In the presence of cytosolic DNA, cGAS generates cGAMPs that bind to STING present intracellularly on the endoplasmic reticulum. This binding induces a confirmational change and downstream cascade involving TBK1 and IRF3 leading to transcription of type I IFNs. Presence of type I IFNs has been correlated with infiltration of activated CD8⁺ T-cells and improved prognostic outcome. Some cancers have defects in the cGAS/STING pathway cultivating an immunosuppressive tumor microenvironment. Strategies to overcome immunosuppression have led to the development of cyclic dinucleotides (CDNs) that act as cGAMP agonist for STING leading to downstream proinflammatory activation. Initial STING agonists were natural CDNs derived from bacterial or human sources, however they were impermeable to cell membranes, had low therapeutic windows, and low tumor bioavailability. In recent years synthetic CDNs and non-CDNs have been developed to overcome barriers associated with natural CDNs and are currently in clinical trials. A phase I clinical trial with 40 patients with advanced metastatic solid tumors or lymphomas reported no dose-limiting toxicities for intratumoral Aduro Biotech STING agonist, ADU-S100. Clinical trials with ADU-S100 and ICB have shown no toxicity, however limited improvement over monotherapy has led to the termination of three phase I/II trials. These trials cannot be directly compared, but the results raise the question of the extent to which STING agonist synergizes with ICB and if alternative combinatorial strategies exist.

Data described herein show that intratumoral STING agonists reprogram TAMs from pro-tumor M2-like to anti-cancer M1-like phenotypes and synergize with PARPi leading to 100% complete regression in patient-mimicking GEMM breast cancer models. Further insights into the mechanism of action of PARPi demonstrated cross-talk between PARPi and the TME related to cGAS-STING/TBK1/IRF3 pathway activation in cancer cells that governs CD8⁺ T-cell recruitment and anti-tumor efficacy, providing rationale for further investigation into the combination.

A key limitation to translation of STING agonist is the need for recurrent intratumoral injections to achieve high disease site drug dose and limit systemic toxicity. Chronic activation of the STING pathway is linked to an autoinflammatory condition, STING-associated vasculopathy with onset in infancy (SAVI). Recently, mutations of STING (G166E) have also been associated with adult-onset autoimmune cutaneous lupus. Given that all cells have some degree of cGAS/STING sensing pathway, intratumoral delivery of STING agonist has been the primary focus of development due to concerns of systemic STING inflammatory activation. A few synthetic CDNs are being assessed for systemic dissemination in phase I clinical trials but results of toxicity are yet to be determined. A preclinical study evaluating the biodistribution and inflammatory responses associate with systemic STING agonist revealed relatively short half-lives for intravenous free and nanoencapsulated STING agonist (2 mins vs 1.3 h, respectively), low tumor specific accumulation (1-3% injected dose), and liver and spleen dose limitations due to organ necrosis and increased serum type I IFNs. The majority of STING agonists under clinical investigation are being evaluated for intratumoral delivery in combination with ICBs.

Given the promising preliminary results of intratumoral STING agonist and PARPi rapid clinical translation is necessary, however hurdles of STING agonist delivery must be overcome. Sustained release polymer-drug formulations are promising alternatives to recurrent intratumoral injections demonstrating partial tumor regression in head and neck and glioblastoma murine models. In both cases, sustained STING agonist release improved tumor regression and increased overall survival compared to periodic free drug. One limitation with the published platforms is their short 1-5 day release kinetics.

Described herein is a formulation sustained release SMAART (Sustained Modulation and Activation of Anticancer Responses and Targets) implants from FDA approved poly(lactic-co-glycolic) acid (PLGA) polymer that have zero-order release kinetics over 28-days and prolong duration of drug action. Release kinetics were demonstrated with TLZ and significantly reduced tumors in BRCA-deficient ovarian cancer models compared to free drug.

Described herein are methods to formulate a platform for sustained intratumoral STING pathway activation and evaluate its impact at primary and secondary, metastatic sites. The formulations described herein are tested to study the tumor immune microenvironment shifts following sustained versus periodic STING pathway activation, how primary site cGAS/STING activation impacts the immune microenvironment at secondary, metastatic sites, and the synergistic impact of sustained STING pathway activation with PARPi to eradicate advanced breast cancer.

Described herein are methods of treating cancer in a subject in need thereof, the method comprising administering to a tumor site of the subject an effective amount of the depot, disclosed herein.

As used herein, the terms “administering” and “administration” refer to any method of providing a pharmaceutical preparation to a subject. Methods of administering the depot are intratumoral or intertumoral injections. Methods of administering additional therapeutics (e.g., an antibody) are well known to those skilled in the art and include, but are not limited to, intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The depot may be administered every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, or every 20 weeks.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

In some embodiments, the cancer is a solid tumor, such as breast cancer (e.g., triple-Negative Breast Cancer and advanced breast cancer), ovarian cancer, colon cancer, lung cancer, prostate cancer, colorectal cancer, pancreatic cancer, liver cancer, kidney cancer, cervical cancer, esophageal cancer, osteosarcoma, chondrosarcoma, liposarcoma, leiomyosarcoma, rhabdomyosarcoma, glioblastoma, meningioma, medulloblastoma, astrocytoma, ependymoma, Hodgkin lymphoma, or non-Hodgkin lymphoma.

The method may further comprise administering an additional therapeutic, such as an anti-PDL1 antibody, a STING agonist, and/or a PARPi, optionally formulated in a liposome. The liposome may comprise DSPE-PEG-amine (1,2-Distearoyl-sn-glycero-3-phosphoethanolamine-polyethylene glycol-amine) and/or DSPE-PEG-COOH (1,2-Distearoyl-sn-glycero-3-phosphoethanolamine-polyethylene glycol-carboxyl), DOTAP (1,2-Dioleoyl-3-trimethylammonium-propane), cholesterol, and DPPC (1,2-Dipalmitoyl-sn-glycero-3-phosphocholine). The additional therapeutic may be. administered every week, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 14 weeks, every 15 weeks, every 16 weeks, every 17 weeks, every 18 weeks, every 19 weeks, or every 20 weeks. In some embodiments, the liposome is 50 nm, 55 nm, 60 nm, 65 nm, 70 nm, 75 nm, 80 nm, 85 nm, 90 nm, 95 nm, or 100 nm in size.

### Anti-PD-L1 Antibody Sequences

| | | |
|---|---|---|
| SEQ ID NO | Description | Sequence |

Atezolizumab

| | | |
|---|---|---|
| 1 | PD-L1 | GFTFSDSWIH |
| | CDR-H1 | |
| 2 | PD-L1 | AWISPYGGSTYYADSVKG |
| | CDR-H2 | |
| 3 | PD-L1 | RHWPGGFDY |
| | CDR-H3 | |
| 4 | PD-L1 | RASQDVSTAVA |
| | CDR-L1 | |
| 5 | PD-L1 | SASFLYS |
| | CDR-L2 | |
| 6 | PD-L1 | QQYLYHPAT |
| | CDR-L3 | |
| 7 | PD-L1 VH | EVQLVESGGGLVQPGGSLRLSCAASGFTFSDSWIHWVR |
| | | QAPGKGLEWVAWISPYGGSTYYADSVKGRFTISADTS |
| | | KNTAYLQMNSLRAEDTAVYYCARRHWPGGFDYWGQ |
| | | GTLVTVSS |
| 8 | PD-L1 VL | DIQMTQSPSSLSASVGDRVTITCRASQDVSTAVAWYQQ |
| | | KPGKAPKLLIYSASFLYSGVPSRFSGSGSGTDFTLTISSL |
| | | QPEDFATYYCQQYLYHPATFGQGTKVEIK |
| 9 | PD-L1 HC | EVQLVESGGGLVQPGGSLRLSCAASGFTFSDSWIHW |
| | | VRQAPGKGLEWVAWISPYGGSTYYADSVKGRFTIS |
| | | ADTSKNTAYLQMNSLRAEDTAVYYCARRHWPGGF |
| | | DYWGQGTLVTVSSASTKGPSVFPLAPSSKSTSGGTAA |
| | | LGCLVKDYFPEPVTVSWNSGALTSGVHTFPAVLQSSGL |
| | | YSLSSVVTVPSSSLGTQTYICNVNHKPSNTKVDKKVEP |
| | | KSCDKTHTCPPCPAPELLGGPSVFLFPPKPKDTLMISRT |
| | | PEVTCVVVDVSHEDPEVKFNWYVDGVEVHNAKTKPR |
| | | EEQYASTYRVVSVLTVLHQDWLNGKEYKCKVSNKAL |
| | | PAPIEKTISKAKGQPREPQVYTLPPSREEMTKNQVSLTC |
| | | LVKGFYPSDIAVEWESNGQPENNYKTTPPVLDSDGSFF |
| | | LYSKLTVDKSRWQQGNVFSCSVMHEALHNHYTQKSLS |
| | | LSPGK |
| 10 | PD-L1 LC | DIQMTQSPSSLSASVGDRVTITCRASQDVSTAVAWY |
| | | QQKPGKAPKLLIYSASFLYSGVPSRFSGSGSGTDFTL |
| | | TISSLQPEDFATYYCQQYLYHPATFGQGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGECGGGGGGGC |
| | | VIM |

Avelumab

| | | |
|---|---|---|
| 11 | PD-L1 | SYIMM |
| | CDR-H1 | |
| 12 | PD-L1 | SIYPSGGITFYADTVKG |
| | CDR-H2 | |
| 13 | PD-L1 | IKLGTVTTVDY |
| | CDR-H3 | |
| 14 | PD-L1 | TGTSSDVGGYNYVS |
| | CDR-L1 | |
| 15 | PD-L1 | DVSNRPS |
| | CDR-L2 | |
| 16 | PD-L1 | SSYTSSSTRV |
| | CDR-L3 | |
| 17 | PD-L1 VH | EVQLLESGGGLVQPGGSLRLSCAASGFTFSSYIMMWVR |
| | | QAPGKGLEWVSSIYPSGGITFYADTVKGRFTISRDNSKN |
| | | TLYLQMNSLRAEDTAVYYCARIKLGTVTTVDYWGQG |
| | | TLVTVSS |
| 18 | PD-L1 VL | QSALTQPASVSGSPGQSITISCTGTSSDVGGYNYVSWY |
| | | QQHPGKAPKLMIYDVSNRPSGVSNRFSGSKSGNTASLT |
| | | ISGLQAEDEADYYCSSYTSSSTRVFGTGTKVTVL |

Durvalumab

| | | |
|---|---|---|
| 19 | PD-L1 | GFTFSRYWMS |
| | CDR-H1 | |
| 20 | PD-L1 | NIKQDGSEKYYVDSVKG |
| | CDR-H2 | |
| 21 | PD-L1 | EGGWFGELAFDY |
| | CDR-H3 | |
| 22 | PD-L1 | RASQRVSSSYLA |
| | CDR-L1 | |
| 23 | PD-L1 | DASSRAT |
| | CDR-L2 | |
| 24 | PD-L1 | QQYGSLPWT |
| | CDR-L3 | |
| 25 | PD-L1 VH | EVQLVESGGGLVQPGGSLRLSCAASGFTFSRYWMSWV |
| | | RQAPGKGLEWVANIKQDGSEKYYVDSVKGRFTISRDN |
| | | AKNSLYLQMNSLRAEDTAVYYCAREGGWFGELAFDY |
| | | WGQGTLVTVSS |
| 26 | PD-L1 VL | EIVLTQSPGTLSLSPGERATLSCRASQRVSSSYLAWYQQ |
| | | KPGQAPRLLIYDASSRATGIPDRFSGSGSGTDFTLTISRL |
| | | EPEDFAVYYCQQYGSLPWTFGQGTKVEIK |

Cosibelimab

| | | |
|---|---|---|
| 27 | PD-L1 VH | EVQLVQSGAEVKKPGSSVKVSCKASGGTFSRSAISWVR |
| | | QAPGQGLEWMGVIIPAFGEANYAQKFQGRVTITADEST |
| | | STAYMELSSLRSEDTAVYYCARGRQMFGAGIDFWGQG |
| | | TLVTVSS |
| 28 | PD-L1 VL | NFMLTQPHSVSESPGKTVTISCTRSSGSIDSNYVQWYQ |
| | | QRPGSAPTTVIYEDNQRPSGVPDRFSGSIDSSSNSASLTI |
| | | SGLKTEDEADYYCQSYDSNNRHVIFGGGTKLTVL |

### EXAMPLES

### Example 1: Sustained Release Implants can be Easily Fabricated with Various Drugs, Different Tailored Release Profiles and a Range of Loading Efficiencies

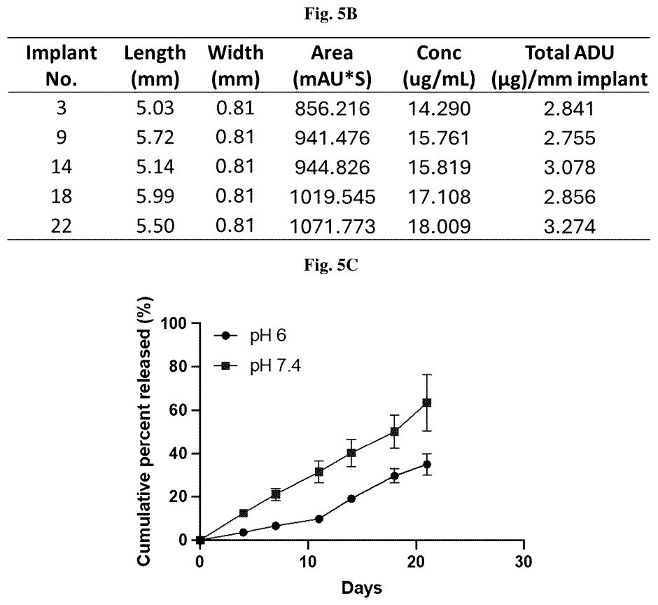

PLGA, an FDA approved polymer, biodegradable sustained release implants (SMAART—Sustained Modulation and Activation of Anticancer Responses and Targets) has been formulated to maintain long-term tumoral drug concentration within the appropriate therapeutic window (FIGS. 5A-5B). The implant was designed to allow direct placement into breast tumors using an 18G needle, after which drug is slowly released by PLGA degradation primarily due to hydrolysis and allowed to diffuse into the tumor. Ease of modulator implant fabrication has been demonstrated across three drugs (FIGS. 5C-5D) with tailored release profiles (30-60 days) and loading efficiencies (25-250 μg mm-1).

### Example 2: Sustained Drug Release Improves Therapeutic Efficacy Compared to Periodic Delivery

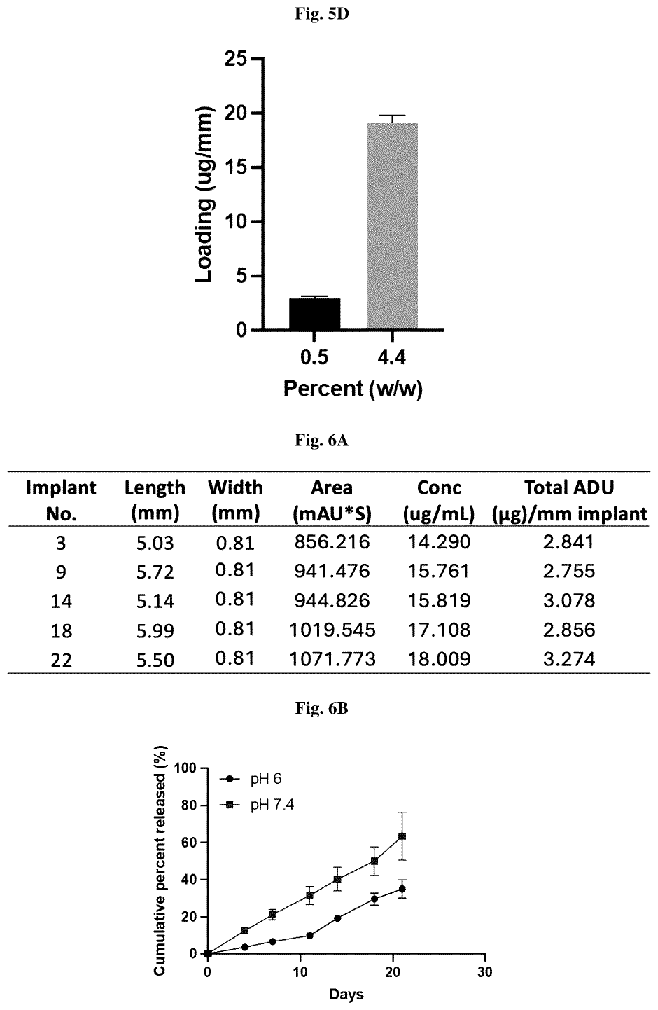

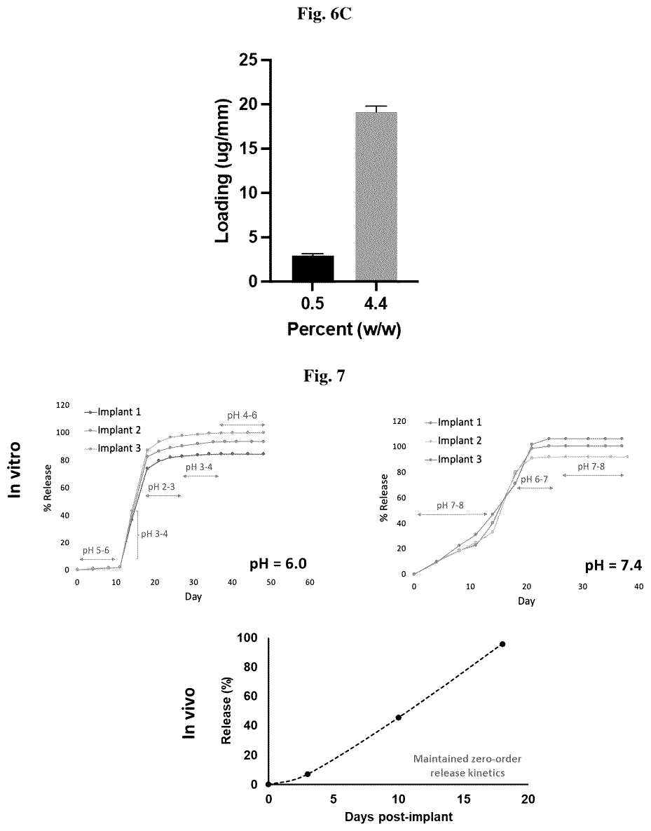

In a Brca2^{-/-}p53^{-/-R172H} Pten^{-/-} intraperitoneal disseminated metastatic ovarian cancer model, SMAART-TLZ i.p implants (50 μg TLZ release over 21 days) increased median survival to 92 days compared to periodic i.p injections of TLZ at the equivalent drug dose over 21 days (70 days median OS-FIG. 6A). Western blots confirmed expression of key markers related to PARP inhibition demonstrating an increase in cleaved caspase 3, an apoptotic marker, and γ-H2AX, a marker of DNA damage, after SMAART-TLZ treatment (FIG. 6B). No increase was observed in empty implants confirming that cellular damage is due to TLZ release.

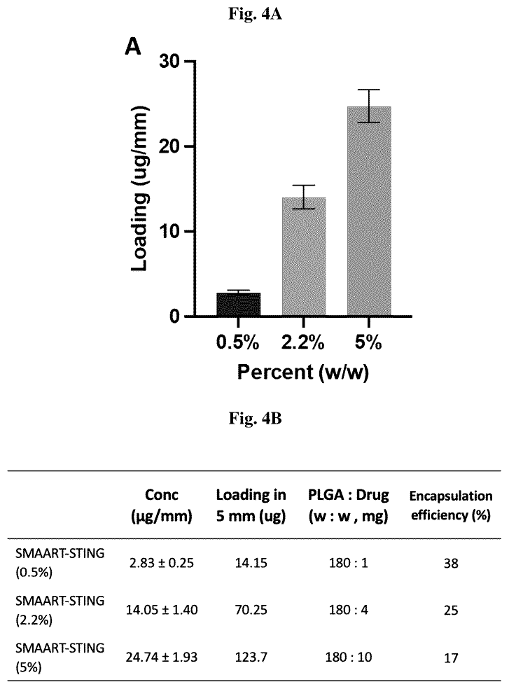

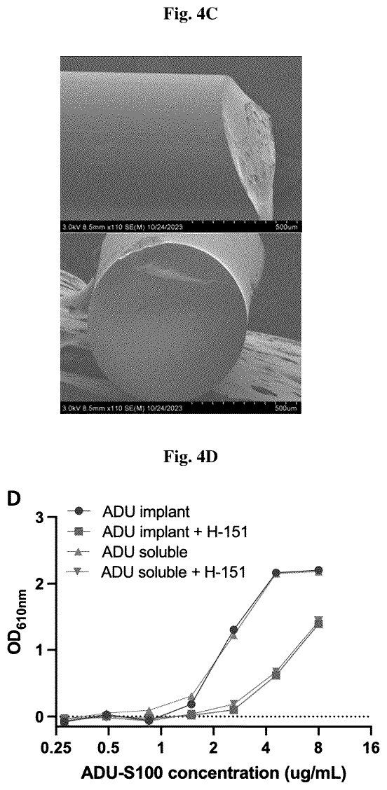

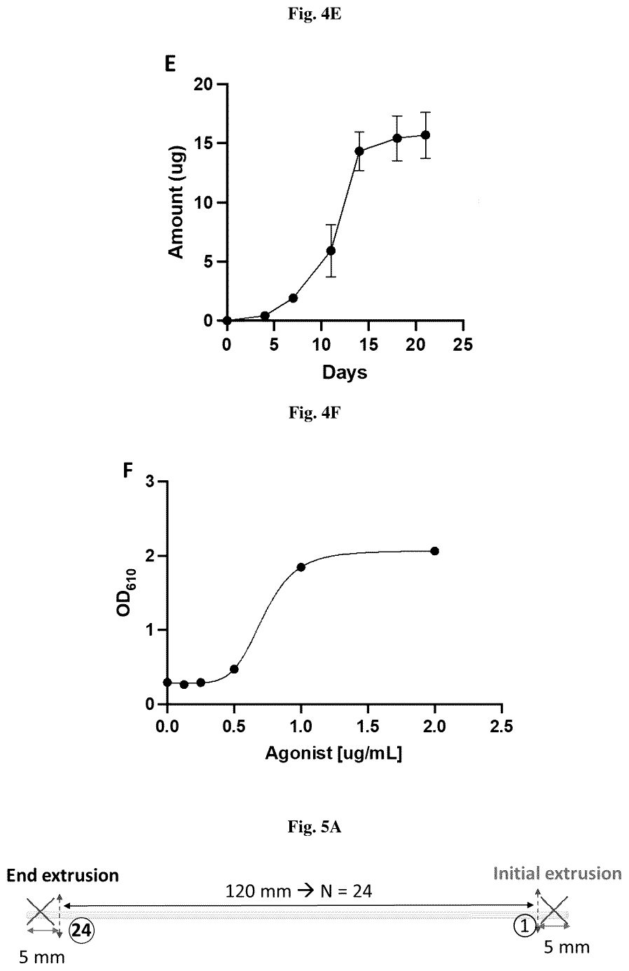

FIGS. 4A-4F show 50:50 PLGA+ADU-S100 sodium. FIG. 4A shows drug loading of first generation SMAART-STING implants at three different weight ratios confirms linearity of final loading compared to initial polymer: drug ratio using 50:50 PLGA and ADU-$100 disodium salt. FIG. 4B shows encapsulation efficacy of SMAART implants decreased with increasing ratios of polymer: drug due to increase in suspension viscosity. FIG. 16C shows SEM of a blank 50:50 PLGA implant. FIG. 4D shows THP-1 reporter cell line confirms that implant loaded ADU-S100 maintains bioactivity and this activity is STING specific (H-151, STING inhibitor). FIG. 4E shows SMAART-STING sustained release kinetics in PBS (pH 7.4, 37° C.). FIG. 4F shows released drug at day 11 maintains bioactivity.

FIGS. 5A-5D show solvent evaporation method with 75:25 PLGA+ADU-$100 sodium. Solvent evaporation method-Loading map (1 mg start). If 100% loading=8.33 μg/mm. Actual loading 35.53%=>2.961+/−0.212 μg. Solvent evaporation method-Loading map (8 mg start-scale-up). If 100% loading=66.67 μg/mm. Actual loading 29.93%=>19.183+/−0.535 μg. FIG. 5A shows implanting mapping example of extruded tubing cut and selected pieces were evaluated via HPLC for homogeneity. FIG. 5B shows solvent evaporation implant formulation mapping for SMAART-STING using 75:25 ratio PLGA and ADU-S100 enantiomer ammonium salt. FIG. 5C shows sustained release of 75:25 PLGA SMAART-STING loaded with ADU-S100 enantiomer ammonium salt in PBS (pH 7.4, pH 6 and 37° C.). FIG. 5D shows drug loading of third generation SMAART-STING implants at two different weight ratios confirms linearity of final loading compared to initial polymer: drug ratio using 75:25 PLGA and ADU-S100 enantiomer ammonium salt.

FIGS. 6A-6C show hot melt extrusion method with 75:25 PLGA+ADU-S100 sodium/ammonium. Solvent evaporation method-Loading map (1 mg start). If 100% loading=8.33 μg/mm. Actual loading 35.53%=>2.961+/−0.212 μg. Solvent evaporation method-Loading map (8 mg start-scale-up). If 100% loading=66.67 μg/mm. Actual loading 29.93%=>19.183+/−0.535 μg. FIG. 6A shows hot melt extrusion implant formulation mapping for SMAART-STING using 50:50 ratio PLGA and ADU-S100 enantiomer ammonium salt. FIG. 6B shows sustained release of 50:50 PLGA SMAART-STING loaded with ADU-S100 enantiomer ammonium salt in PBS (37° C.) at pH 6 and pH 7.4. SE=solvent evaporation sample. Sample X=individual hot-melt extrusion samples. FIG. 6C shows drug loading of third generation SMAART-STING implants at two different weight ratios confirms linearity of final loading compared to initial polymer: drug ratio using 75:25 PLGA and ADU-S100 enantiomer ammonium salt via hot melt extrusion.

FIG. 7 shows solvent evaporation method with 75:25 PLGA+ADU-S100 sodium. In vitro sustained release of 75:25 PLGA:ADU-S100 implants via solvent based evaporation at pH 6 and pH 7 media in vitro. Additional analysis of media showed a change of pH during the hydrolysis and degradation of implant over time. In vivo release study in healthy mice with a subcutaneous implant placed over 21 days showed a zero-order release kinetics.

FIG. 8 shows THP-1 reporter cell line confirms that implant loaded ADU-S100 maintains bioactivity and this activity is STING specific (H-151, STING inhibitor).

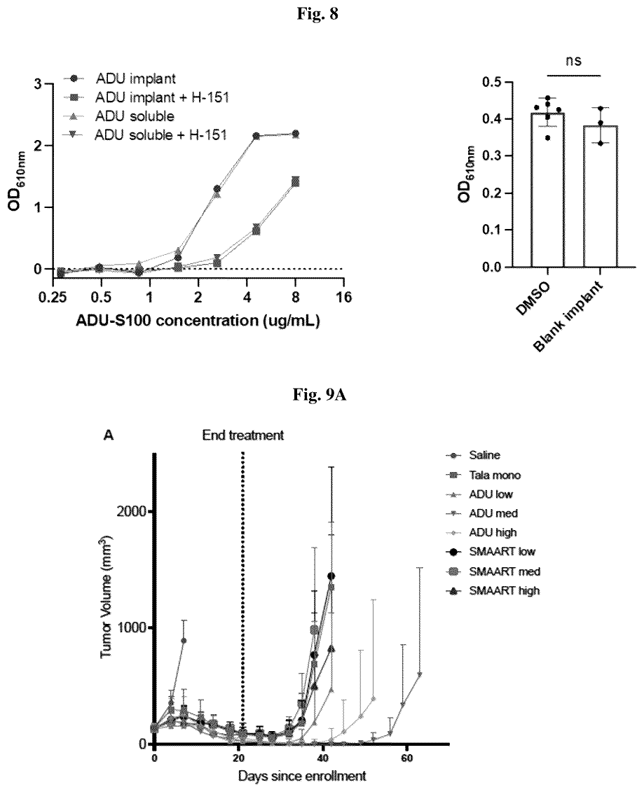

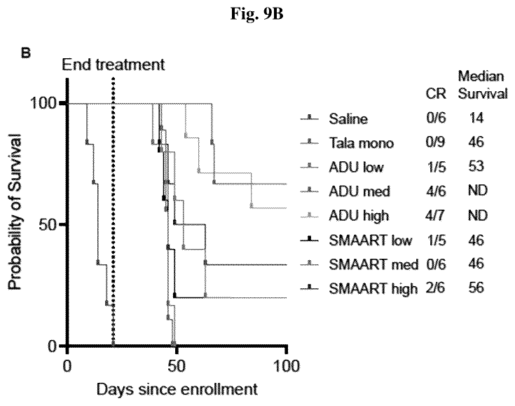

FIGS. 9A-9B show that a TLZ dose study was conducted to identify a drug dose that would allow for the assessment of combinatorial approaches with soluble or SMARRT formulated ADU-S100 with TLZ shown as either FIG. 9A tumor volume or FIG. 9B overall survival.

### Example 3: Synthesis of Anti-PDL1 Nanoparticles which Synergizes with InCeT-TLZ-Dependent Immune Activation

(1) Synthesis and Characterization of a STING Agonist and Anti-PDL1 Nanoformulation to Synergize with InCeT-TLZ

Nanoformulation of ADU-S100, a STING agonist, and anti-PDL1 increase bioavailability of the immune modulators in metastatic ovarian cancer.

Nanoparticles are formulated via a nanoprecipitation technique using a benchtop NanoAssemblr following previously established lab protocols. ADU-S100 is combined with DPPC, DOTAP, cholesterol, and DSPE-PEG-Carboxyl to form liposomes approximately 75 nm. As previously described, EDC/NHS click chemistry is used to surface modify NanoSTING liposomes with anti-PDL1. Nanoformulations are characterized via DLS, zeta-potential, and electron microscopy to determine size and surface charge. ADU-S100 loading efficacy and release kinetics are measured via HPLC-MS and anti-PDL1 binding is confirmed via ELISA. Appropriate controls of empty nanoformulations and IgG surface modified nanoformulations are included. Nanoparticle internalization is measured on BPPNM and mFT3666 cells overexpressing PDL1 and appropriate controls of blocking with free anti-PDL1 and IgG are added.

### (2) Nanoformulations of a STING Agonist and Anti-PDL1 Facilitate T-Cell Infiltration in Tumors.

A STING agonist and anti-PDL1 nanoformulation favorably increases infiltration of pro-tumor immune cells compared to free drugs.

### INCORPORATION BY REFERENCE

Each of the patents, published patent applications, and non-patent references cited herein are hereby incorporated by reference in their entirety.

### EQUIVALENTS

Those skilled in the art will recognize, or be able to ascertain using no more than routine experimentation, many equivalents to the specific embodiments described herein. Such equivalents are intended to be encompassed by the following claims.

1. A depot, comprising a biodegradable polymer and a STING agonist or a PARP inhibitor (PARPi); wherein the STING agonist or the PARPi is distributed in the biodegradable polymer.

2. The depot of claim 1, wherein the depot is configured to be implanted at a tumor site of a patient.

3. The depot of claim 1, wherein the depot releases the STING agonist or the PARPi at the tumor site for a period of time.

4. The depot of claim 3, wherein the period of time is no less than 15 days.

5. The depot of claim 3, wherein the period of time is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days.

6. The depot of claim 2, wherein the depot is configured to be delivered to the tumor site through a needle.

7. The depot of claim 6, wherein the needle is an 18G needle.

8. The depot of claim 1, wherein the biodegradable polymer comprises polyglycolide (PGA), polycaprolactone (PCL), poly(DL-lactic acid) (PLA), poly(alpha-hydroxy acids), poly(lactide-co-glycolide) (PLGA or DLG), poly(DL-lactide-co-caprolactone) (DL-PLCL), poly(trimethylene carbonate) (PTMC), polydioxanone (PDO), poly(4-hydroxy butyrate) (PHB), polyhydroxyalkanoates (PHA), poly(phosphazene), polyphosphate ester), poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (such as hyaluronic acid, chitosan and starch), proteins (such as gelatin and collagen) or PEG derivatives, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or poly(DL-lactide-co-glycolide-co-caprolactone).

9. The depot of claim 1, wherein the polymer is poly(DL-lactide-co-glycolide) (PLGA).

10. The depot of claim 9, wherein the PLGA comprises equal parts lactide and glycolide.

11. The depot of claim 9, wherein said PLGA comprises 75% lactide and 25% glycolide, or 85% lactide and 15% glycolide.

12. The depot of claim 1, wherein the depot comprises 1 mg to 10 mg, 10 mg to 20 mg, 20 mg to 30 mg, 30 mg to 40 mg, 40 mg to 50 mg, 50 mg to 60 mg, 60 mg to 70 mg, 70 mg to 80 mg, 80 mg to 90 mg, 90 mg to 100 mg, 100 mg to 110 mg, 110 mg to 120 mg, 120 mg to 130 mg, 130 mg to 140 mg, 140 mg to 150 mg, 150 mg to 160 mg, 160 mg to 170 mg, 170 mg to 180 mg, 180 mg to 190 mg, 190 mg to 200 mg, 200 mg to 210 mg, 210 mg to 220 mg, 220 mg to 230 mg, 230 mg to 240 mg, 240 mg to 250 mg, 250 mg to 260 mg, 260 mg to 270 mg, 270 mg to 280 mg, 280 mg to 290 mg, 290 mg to 300 mg, 300 mg to 310 mg, 310 mg to 320 mg, 320 mg to 330 mg, 330 mg to 340 mg, 340 mg to 350 mg, 350 mg to 360 mg, 360 mg to 370 mg, 370 mg to 380 mg, 380 mg to 390 mg, or 390 mg to 400 mg of the STING agonist or the PARPi.

13. The depot of claim 1, wherein the depot comprises both the STING agonist and the PARPi.

14. The depot of claim 1, wherein the STING agonist stimulates an anticancer immune microenvironment.

15. The depot of claim 1, wherein the STING agonist activates anticancer innate and adaptive immunity within the tumor microenvironment.

16. The depot of claim 13, wherein the STING agonist and the PARPi promote immune infiltration of secondary, metastatic sites.

17. The depot of claim 13, wherein the STING agonist amplifies the antitumor efficacy of the PARPi.

18. The depot of claim 1, wherein the STING agonist is MK-1454, ADU-S100, GSK3745417, SB 11285, DMXAA, MPLA (Monophosphoryl Lipid A), a Cyclic Dinucleotide (CDN), E7766, BMS-986301, or Astin C.

19. The depot of claim 1, wherein the PARPi is Olaparib, Niraparib, and Talazoparib (TLZ), Rucaparib, Veliparib, Pamiparib, or Fuzuloparib.

20. (canceled)

21. A method of treating cancer in a subject in need thereof, comprising administering to a tumor site of the subject an effective amount of the depot of claim 1.

22.-42. (canceled)


SEQUENCE LISTING (28 sequences)

SEQ ID NO: 1 - protein, 10 aa, synthetic construct
GFTFSDSWIH

SEQ ID NO: 2 - protein, 18 aa, synthetic construct
AWISPYGGSTYYADSVKG

SEQ ID NO: 3 - protein, 9 aa, synthetic construct
RHWPGGFDY

SEQ ID NO: 4 - protein, 11 aa, synthetic construct
RASQDVSTAVA

SEQ ID NO: 5 - protein, 7 aa, synthetic construct
SASFLYS

SEQ ID NO: 6 - protein, 9 aa, synthetic construct
QQYLYHPAT

SEQ ID NO: 7 - protein, 118 aa, synthetic construct [sequence omitted]

SEQ ID NO: 8 - protein, 107 aa, synthetic construct [sequence omitted]

SEQ ID NO: 9 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 225 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGGGCVIM

SEQ ID NO: 11 - protein, 5 aa, synthetic construct
SYIMM

SEQ ID NO: 12 - protein, 17 aa, synthetic construct
SIYPSGGITFYADTVKG

SEQ ID NO: 13 - protein, 11 aa, synthetic construct
IKLGTVTTVDY

SEQ ID NO: 14 - protein, 14 aa, synthetic construct
TGTSSDVGGYNYVS

SEQ ID NO: 15 - protein, 7 aa, synthetic construct
DVSNRPS

SEQ ID NO: 16 - protein, 10 aa, synthetic construct
SSYTSSSTRV

SEQ ID NO: 17 - protein, 120 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 18 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 19 - protein, 10 aa, synthetic construct
GFTFSRYWMS

SEQ ID NO: 20 - protein, 17 aa, synthetic construct
NIKQDGSEKYYVDSVKG

SEQ ID NO: 21 - protein, 12 aa, synthetic construct
EGGWFGELAFDY

SEQ ID NO: 22 - protein, 12 aa, synthetic construct
RASQRVSSSYLA

SEQ ID NO: 23 - protein, 7 aa, synthetic construct
DASSRAT

SEQ ID NO: 24 - protein, 9 aa, synthetic construct
QQYGSLPWT

SEQ ID NO: 25 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 26 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 27 - protein, 120 aa, synthetic construct
EVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSS

SEQ ID NO: 28 - protein, 112 aa, synthetic construct
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVL